(19) United States
(12) Patent Application Publication
(10) Pub. No.: US 2002/0064875 A1
(43) Pub. Date: May 30, 2002

(54) Carrier for cell attachment or fixation and its process for preparation

(75) Inventors: Chun-Chieh Chen, Tainan (TW); Yu-Tai Liu, Hsinchu (TW); King-Ming Chang, Hsinchu (TW); Chen-Ze Hu, Chung-Ho (TW)

Correspondence Address: LADAS & PARRY, 26 WEST 61ST STREET, NEW YORK, NY, 10023, US

(73) Assignee: INDUSTRIAL TECHNOLOGY RESEARCH INSTITUTE

(21) Appl. No.: 09/923,130

(22) Filed: Aug. 6, 2001

(30) Foreign Application Priority Data
Oct. 6, 2000 (TW) 89120904

(51) Int. Cl. C12N 5/02; B32B 27/12

(52) U.S. Cl. 435/399; 442/123

(57) ABSTRACT

The present invention discloses a carrier for cell attachment or fixation and its preparation. The carrier of the present invention is formed by the following steps: forming a fiber by extruding a melted polymer from a nozzle: extending the fiber and shaping the extended fiber on the shaping net to form a three-dimensional branch-like non-woven structure; activating the surface of the non-woven structure to have cell affinity and facilitate the attaching and growing of the cell. Moreover, the surface of the carrier is wrinkled or rough to improve the stiffness of the carrier and keep enough space between the carriers.

### BACKGROUND OF THE INVENTION

1. Field of the Invention

The present invention relates to a carrier and its preparation process, and more particularly, to a carrier for cell attachment or fixation in cell culture.

2. Description of the Prior Art

With the development of biotechnology, the culture technique of animal cells becomes more and more important and its market value is increasing as well. Except for the cell suspension culture process, the cells are cultivated by attaching on the carrier in general processes because animal cells have the following characteristics: slow to grow, easy to be harmed by shear stress and contaminated by microorganisms, costly and risky to cultivate and most animal cells have anchorage-dependent properties. The carrier for cell culture is a micro-carrier, a porous ceramic carrier, a fiber carrier or a hollow fiber carrier, etc.

Conventional carriers for cell attachment can be classified into two types: the first type is a particle carrier with smooth surface on which the cells can only grow; the second type is a porous carrier in which the cells can grow deeply inside. The cells growing on the first type of carrier are easy to detach and recover. But, the surface area for cell attachment of the first type of carrier cannot be increased because the surface of the carrier is smooth. The second type of carrier can provide larger surface area for cell attachment; however, the cells growing in this carrier can hardly be retrieved because they grow deeply inside.

U.S. Pat. No. 5,266,476 discloses a carrier for cell culture made by a fiber. The carrier is a double-layered structure that is composed of a cut fiber and a polymer-meshed net. The carrier provides a larger ratio of the surface area to the volume so as to increase the density of the cultivated cell. Moreover, an adequate nutrient is supplied in the carrier of thin sheet so that the cell growing inside would not die due to the lack of oxygen or nutrition. In order to improve the stiffness of the carrier, it is necessary to combine a cut fiber with a polymer meshed net and form a double-layered structure of the carrier. Therefore, the manufacture process of the carrier is complicated and costly and the ratio of the surface area to the volume of the carrier would be decreased because of the double-layered structure of the carrier. Moreover, the fiber of the polymer meshed net is formed by ordinary spinning processing, and a residual spinning oil is a pollution and needs post-treatment.

In order to prevent the disadvantages of conventional carriers, it is necessary to provide a novel carrier for cell culture. The present invention provides a carrier with a single layer and a three-dimensional branch-like structure for cell attachment and fixation as well as a simple method for preparing the carrier. The carrier of the present invention is useful in promoting growth of cells.

### SUMMARY OF THE INVENTION

The present invention discloses a carrier for cell attachment and fixation which provides a three-dimensional structure for cell culture, prevents the cell from being harmed by mechanical force and keeps the stability of the grown cell.

In order to achieve the purposes of the invention and prevent the disadvantages of prior art, an object of the present invention is to provide a carrier for cell attachment and fixation formed by following steps: forming a fiber by extruding a melted polymer from a nozzles: extending the fiber and shaping the extended fiber on a shaping net to form a non-woven structure; and activating the surface of the non-woven structure to have cell affinity. The fiber of the non-woven structure is inter-connected and constructed as a three-dimensional branch-like structure. Moreover, the fiber of the non-woven structure facilitates the cell attaching and growing.

Another object of the present invention is to provide a carrier with a single layer. Therefore, the method for preparing the carrier is simplified and the spinning oil or any other additive is not used during manufacture; by doing so further possibilities of pollution is avoided.

The second object of the present invention is to provide a non-woven carrier with a wrinkled or rough surface to improve the stiffness of the carrier. Therefore, the space between the carriers can be kept when the carriers are filled into a packed bed.

The foregoing and other objects and advantages of the invention and the manner in which the same are accomplished will become clearer based on the following detailed description taken in conjunction with the accompanying drawings.

### BRIEF DESCRIPTION OF THE DRAWINGS

FIG. 1 depicts a SEM diagram of the carrier with a wrinkled single layer of the present invention which is magnified for 20 times;

FIG. 2 depicts a SEM diagram of the carrier with a branch-like structure of the present invention which is magnified for 150 times;

FIG. 3 depicts a comparative diagram of the culture effects which are obtained from a PP network carrier of the present invention and a commercial product;

FIG. 4(A) depicts a culture result of the carrier of the present invention, which is weighed on the basis of 50 g/m²;

FIG. 4(B) depicts a culture result of the carrier of the present invention, which is weighed on the basis of 30 g/m²;

FIG. 5(A) depicts culture results of the cells cultivated on the PP carrier with a three-dimensional branch-like structure of the present invention with the use of a serum.

FIG. 5(B) depicts culture results of the cells cultivated on the PP carrier with a three-dimensional branch-like structure of the present invention without the use of a serum.

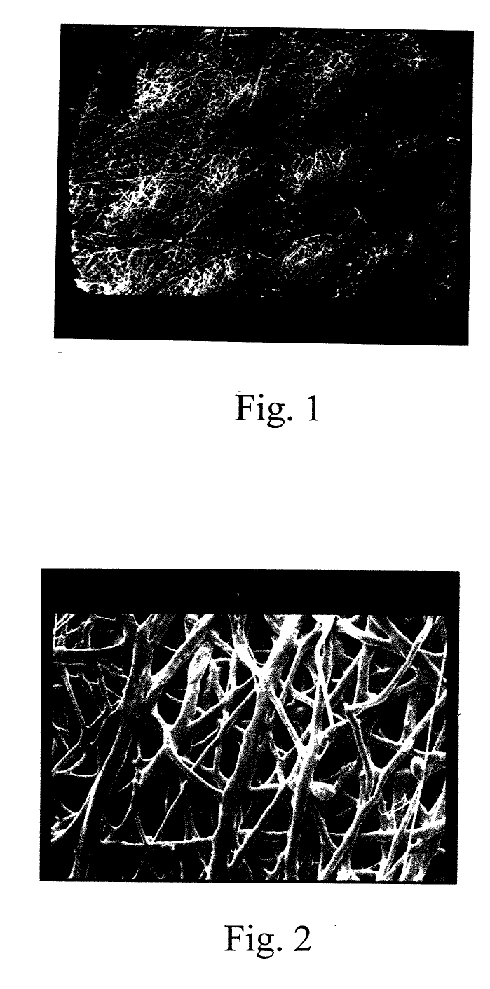

### DETAILED DESCRIPTION OF THE INVENTION

In order to prevent the disadvantages of the prior art, the present invention discloses a non-woven structure carrier which is formed by a polymer melt blowing process that comprises the following steps: forming a fiber by extruding a melted polymer from a nozzle quantitatively; extending the fiber and shaping the extended fiber on a shaping net to form a non-woven structure; and activating the surface of the non-woven structure to have cell affinity. The fiber is extended by a manner of air or machine to have the diameter of between 2 to 15 micrometers. The fiber is a circular or irregular hollow or solid pipe in shape. The porosity of the non-woven structure is between 40% and 90%.

The polymer material is selected from the group that consists of polyethylene, polypropylene, polyurethane, polyester, polyacrylonitrile, polyvinyl acetate compounding, polyvinyl alcohol, polyacetic acid, polyvinylidene chloride, polystyrene, polybutadiene, glass fiber, cellulose, fluorocarbon resin, collagen and the copolymer thereof.

The fiber of the non-woven structure is inter-connected so as to form a three-dimensional branch-like structure. The surface of the fiber of the non-woven structure is treated with an activated grafting treatment to form a carrier with cell affinity and to facilitate the cell attaching and growing.

The thickness of the carrier of the present invention is between 200 and 600 micrometers. The shape of the carrier formed by a cutter or a key punch is a circular, square, polygonal or irregular sheet. The carrier of the present invention is circular and has the diameter of between 2 and 10 mm. The non-woven structure of the invention is a single layer sheet with a wrinkled or rough surface. The wrinkled surface of the non-woven structure can improve the stiffness of the carrier and keep the space between the carriers sufficient to provide oxygen and nutrition for the cell. Moreover, the wrinkled surface of the non-woven structure is useful to simplify the process of manufacture which needs the binding of the fiber and a polymer meshed net to improve the stiffness of the carrier, and avoid the possibility of any further pollution by not using a spinning solvent oil or any other additive during the manufacture process.

The carrier of the invention is formed directly from a non-woven structure with a high porosity treated with a special surface treatment so as to have surface affinity and high surface/volume ratio. The porosity and fiber thickness of the carrier are thus controlled. Moreover, the three-dimensional branch-like continuous structure of the carrier can facilitate the cell attaching and crawling so that the cell growing is improved. In addition, because the surface of the carrier is wrinkled or rough, both sides of the carrier can contact with the culture medium to provide enough nutrition and oxygen for the cell. Therefore, the development of cell culture by using the carrier of the present invention is better than using conventional or commercial carriers.

The present invention also provides a method for preparing the carrier for cell attachment or fixation that comprise the following steps:

(a) forming a fiber by extruding a melted polymer from a nozzle:

(b) extending the fiber and shaping the extended fiber on a shaping net to form a non-woven structure; and

(c) activating the surface of the non-woven structure to have cell affinity.

The method disclosed in the present invention further comprises a step of hot pressing before activating the surface to form a wrinkled surface of the carrier. In addition, the fiber is extended mechanically in a condition of hot air.

Furthermore, the surface of the fiber of the non-woven structure is activated by an activated grafting treatment that comprises the following steps:

(a) activating the surface of the non-woven structure; and

(b) grafting a functional group on the activated surface of the non-woven structure.

In step (a) of the activated grafting treatment, the surface of the non-woven structure is activated by plasma, corona, ultraviolet, radiation or wet type chemistry to improve the cell affinity.

In step (b) of the activated grafting treatment, the activated surface of the non-woven structure is grafted with a functional group by exposing the surface of the non-woven structure in a plasma to a monomer having both an unsaturated functional group and a polar functional group. The unsaturated functional group is selected from a double bond or a triple bond. The compound with the unsaturated functional group is selected from the derivatives of alkyne or alkene. The polar functional group is selected from amino group, carboxylic group, hydroxyl group or sulfonate. The compound with the polar functional group is selected from amine, carboxylic acid, alcohol, sulfonate or the derivatives thereof. Take amine for an example, the amine monomer is bonded to the surface of the activated non-woven structure by breaking the unsaturated bond without destroying the amino group. Moreover, the amino group grafted on the surface by the activated grafting treatment can maintain at least one year in ambient temperature and pressure.

The methods and features of this invention have been sufficiently described in the above descriptions. It should be understood that any modifications or changes without departing from the spirits of the invention are intended to be covered in the protection scope of the invention.

### EXAMPLE 1

MFR 700 polypropylene pellets were melted in an extruding machine and extruded from a nozzle to form a fiber under a condition of 290° C. hot air blowing. The temperature of the nozzle was 280° C. and the diameter of the nozzle was about 0.5 mm. The fiber was extended to have the diameter of the fiber of about 10 micrometers. The extended fiber was then shaped on a shaping net to form a non-woven structure with a three-dimensional brench-like structure, which was weighed on the basis of 50 g/m². The non-woven structure was pressed by a patterned wheel to have a wrinkled surface and punched by a punch machine to form a circular structure with a diameter of about 6 mm. The circular non-woven structure was steamed in methanol for 8 hours and dried at room temperature. The surface of the non-woven structure was activated in plasma machine for 2 mins, in which the ratio of argon to oxygen was 10:1, the flow rate was 500 ml/min, the power was 200 watt and the pressure was 50 mtorr. Subsequently, the activated non-woven structure was grafted under the following conditions to form a carrier.

The pressure of the grafting step: 40 mtorr

| | Power | allylamine | Time |
|---|---|---|---|
| | (W) | (%) | (min) |
| A | 15 | 30 | 5 |
| B | 15 | 30 | 2 |
| C | 15 | 30 | 5 |
| D | 15 | 30 | 8 |
| E | 20 | 30 | 5 |
| F | 15 | 37 | 5 |
| G | 15 | 30 | 5 |
| H | 10 | 30 | 5 |

The carriers treated with the above conditions were dipped in a 99.5% ethanol solution for disinfection for overnight. Each carrier was placed in one well of the prepared 96-well plate. The controlling group of this experiment was commercial Fibra-Cel microcarriers. The experiment under each condition mentioned above was repeated for three times. Then, 10,000 VERO cells (African green monkey kidney cells, ATCC CCL-81) and 200 ul culture medium (M199/5% fetal calf serum) were placed in each well of the plate. The cell was cultured at 37° C., 5% carbon dioxide for 4 days.

After removing the medium on the carrier, a 100 ul MTT (4 mg MTT/10 ml PBS) solution was added to each well of the plate. The cell was cultivated in an incubator (37° C., 5% CO₂) for 3 hours. The MTT solution was then sucked away, and a 100 ul DMSO solution was added to each well of the plate and the plate standed for 20 minutes. A 50 ul reacted solution of each well of the plate was transferred to another 96-well plate and measured by an ELISA reader at a wavelength of 560 nm.

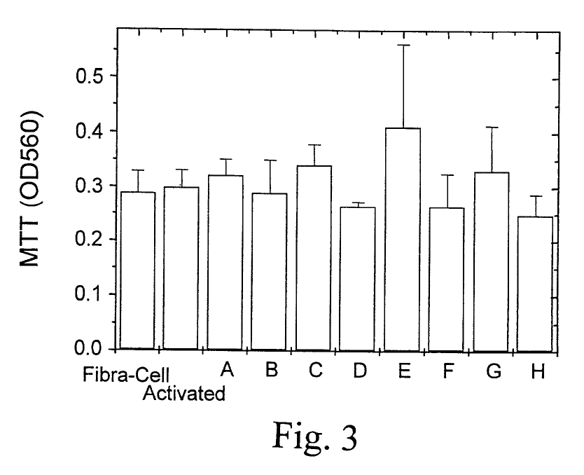

The results of the experiment are shown in FIG. 3 which shows that the PP network carrier of the present invention has better cell culture effect than commercial products.

### EXAMPLE 2

MFR 700 polypropylene pellets were melted in an extruding machine and extruded from a nozzle to form a fiber under a condition of 290° C. hot air blowing. The temperature of the nozzle was 280° C. and the diameter of the nozzle was about 0.5 mm. The fiber was extended to have the diameter of the fiber of about 10 micrometers. The extended fiber was shaped on a shaping net to respectively form a non-woven structure with a three-dimensional branch-like structure, which was weighed on the basis of 50 g/m² (PP-thick) and 30 g/m² (PP-thin). Both non-woven structures were pressed by a patterned wheel to have a wrinkled surface and punched by a punch machine to form a circular structure with a diameter of about 6 mm. The circular non-woven structures were steamed in methanol for 8 hours and dried at room temperature. The surface of both non-woven structures was activated in plasma machine for 2 mins, in which the ratio of argon to oxygen was 10:1, the flow rate was 500 ml/min, the power was 200 watt and the pressure was 50 mtorr. Subsequently, the activated non-woven structures were grafted under the following conditions to form a carrier.

| | A | B | C | D | E |
|---|---|---|---|---|---|
| Power | 15 W | 15 W | 15 W | 15 W | 15 W |
| (W) | | | | | |
| Flow | 500 | 500 | 500 | 500 | 500 |
| (ml/min) | | | | | |
| Allyl | 5% | 10% | 30% | 10% | 10% |
| amine | | | | | |
| Time | 5 | 5 | 5 | 5 | 5 |
| (min) | | | | | |
| Pressure | 40 | 40 | 40 | 80 | 120 |
| (mtorr) | | | | | |

The carriers weighed on the basis of 50 g/m² and 30 g/m² and treated with the above conditions were dipped in a 99.5% ethanol solution for disinfection for overnight. Each carrier was placed in one well of the prepared 96-well plate. The controlling group of this experiment was commercial Fibra-Cel microcarriers. The experiment under each condition mentioned above was repeated for six times. 10,000 VERO cells (ATCC CCL-81) and 200 ul culture medium (M199/5% fetal calf serum) were placed in each well of the plate. The cell was cultured at 37° C., 5% carbon dioxide for 4 days.

After removing the medium on the carrier, a 100 ul MTT (4 mg MTT/10 ml PBS) solution was added to each well of the plate. The cell was cultivated in a CO₂ incubator for 3 hours. The MTT solution was then sucked away, and a 100 ul DMSO solution was added to each well of the plate and the plate standed for 20 minutes. A 50 ul reacted solution of each well of the plate was transferred to another 96-well plate and measured by an ELISA reader at a wavelength of 560 mn.

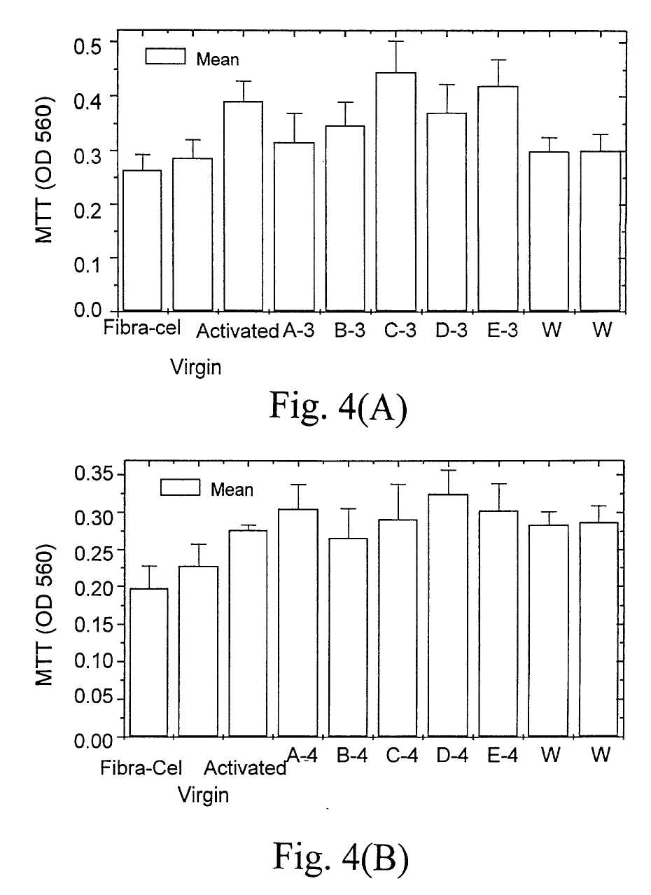

The results of the experiment are shown in FIGS. 4(A) and 4(B). Such figures indicate that the carrier with 50 g/m² has better cell culture effect than the carrier with 30 g/m². Moreover, the culture results of both carriers are better than commercial carriers.

### EXAMPLE 3

MFR 700 polypropylene pellets were melted in an extruding machine and extruded from a nozzle to form a fiber under a condition of 290° C. hot air blowing. The temperature of the nozzle was 280° C. and the diameter of the nozzle was about 0.5 mm. The fiber was extended to have the diameter of the fiber of about 10 micrometers. The extended fiber was shaped on a shaping net to form a non-woven structure with a three-dimensional brench-like structure, which was weighed on the basis of 50 g/m². The non-woven structure was pressed by a patterned wheel to have a wrinkled surface and punched by a punch machine to form a circular structure with a diameter of about 6 mm. The circular non-woven structures were steamed in methanol for 8 hours and dried at room temperature. The surface of the non-woven structures was activated in plasma machine for 2 mins, in which the ratio of argon to oxygen was 10:1, the flow rate was 500 ml/min, the power was 200 watt and the pressure was 50 mtorr. Subsequently, the -activated non-woven structures were grafted under the following conditions to form a carrier.

The pressure of the surface grafting treatment: 60 mtorr

| | Power | Allylacetate | Allylamine | Time |
|---|---|---|---|---|
| | (W) | (%) | (%) | (min) |
| 1 | 20 | 15 | 35 | 2 |
| 2 | 30 | 20 | 35 | 2 |
| 3 | 30 | 20 | 35 | 6 |
| 4 | 30 | 10 | 35 | 6 |
| 5 | 30 | 10 | 35 | 2 |
| 6 | 10 | 10 | 35 | 2 |
| 7 | 10 | 10 | 35 | 6 |
| 8 | 10 | 20 | 35 | 6 |
| 9 | 10 | 20 | 35 | 2 |
| 6' | 10 | 10 | 35 | 2 |
| 7' | 10 | 10 | 35 | 6 |
| 8' | 10 | 20 | 35 | 6 |
| 9' | 10 | 20 | 35 | 2 |

The carriers treated with the above condition were dipped in a 99.5% ethanol solution for disinfection for overnight. Each carrier was placed in one well of the prepared 96-well plate. The controlling group of this experiment was commercial Fibra-Cel microcarriers. The experiment under each condition mentioned above was repeated for six times. 10,000 VERO cells (ATCC CCL-81) and 200 ul culture medium (M199/5% fetal calf serum) or 200 ul serum-free medium (SFCH6000, a self development culture formula) were placed in each well of the plate. The cell was cultured at 37° C., 5% carbon dioxide for 4 days.

After removing the medium on the carrier, a 100 ul MTT (4 mg MTT/10 ml PBS) solution was added to each well of the plate. The cell was cultivated in a CO₂ incubator for 3 hours. The MTT solution was then sucked away, and a 100 ul DMSO solution was added to each well of the plate and the plate standed for 20 minutes. A 50 ul reacted solution of each well of the plate was transferred to another 96-well plate and measured by an ELISA reader at a wavelength of 560 nm.

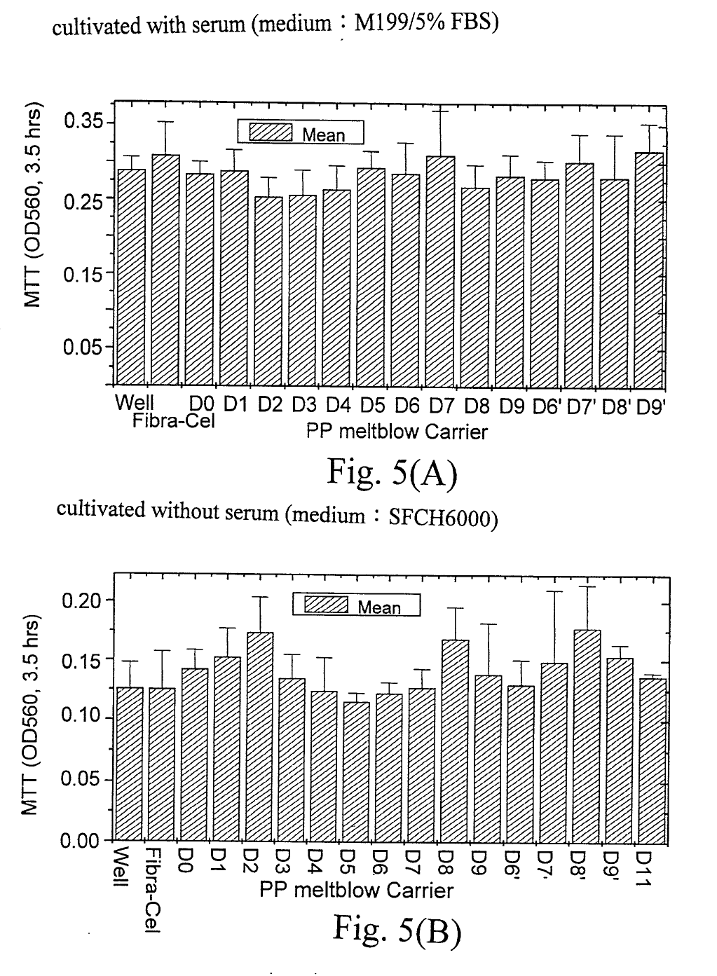

The culture results of the carriers of the present invention and the carriers formed by commercial PET (Fibra-Cel) or PP non-woven structure are shown in FIGS. 5(A), 5(B). The culture results are good of both carriers formed from commercial PET non-woven structure and the carrier formed from PP non-woven structure treated by a melt blowing process. However, in the absence of the serum, the cell cultivated on the carrier of the present invention has better effect because the three-dimensional branch-like continuous structure of the carrier of the present invention improves the cell growth result.

What is claimed is:

1. A carrier for cell attachment or fixation formed by the following steps:
(a) forming a fiber by extruding a melted polymer from a nozzle;
(b) extending the fiber and shaping the extended fiber to form a non-woven structure; and
(c) activating the surface of the non-woven structure to have cell affinity.

2. The carrier of claim 1 being a single layer sheet.

3. The carrier of claim 1, wherein the diameter of the extended fiber is between 2 and 15 micrometers.

4. The carrier of claim 1, wherein the non-woven structure is a three-dimensional branch-like structure.

5. The carrier of claim 1, wherein the porosity of the non-woven structure is between 40% and 90%.

6. The carrier of claim 1, wherein the surface is wrinkled or rough.

7. The carrier of claim 1, wherein the fiber is formed by a melt blowing process.

8. The carrier of claim 1, wherein the carrier is circular, square, polygonal, strip or irregular sheet.

9. The carrier of claim 1, wherein the thickness of the carrier is between 200 and 600 micrometers.

10. The carrier of claim 1, wherein the carrier is circular and has the diameter of between 2 and 10 mm.

11. The carrier of claim 1, wherein the polymer is selected from the group consisting of polyethylene, polypropylene, polyurethane, polyester, polyacrylonitrile, polyvinyl acetate compounding, polyvinyl alcohol, polyactic acid, polyvinylidene chloride, polystyrene, polybutadiene, glass fiber, cellulose, fluorocarbon resin, collagen and the copolymer thereof.

12. The carrier of claim 1, wherein the surface of the non-woven structure in step (c) is treated by an activated grafting treatment to have cell affinity.

13. The carrier of claim 12, wherein the activated grafting treatment comprises the following steps
(a) activating the surface of the non-woven structure; and
(b) grafting a functional group on the activated surface of the non-woven structure.

14. The carrier of claim 13, wherein the surface of the non-woven structure in step (a) is activated by plasma, corona, ultraviolet, radiation or wet type chemistry.

15. The carrier of claim 13, wherein the activated surface of the non-woven structure in step (b) is grafted by exposing the surface of the non-woven structure to a monomer having both an unsaturated functional group and a polar functional group.

16. The method of claim 15, wherein the unsaturated functional group is selected from a double bond or a triple bond.

17. The method of claim 16, wherein a compound with the unsaturated functional group is selected from the derivatives of alkyne or alkene.

18. The method of claim 15, wherein the polar functional group is selected from amino group, carboxylic group, hydroxyl group or sulfonate.

19. The method of claim 18, wherein a compound with the polar functional group is selected from amine, carboxylic acid, alcohol, sulfonate or the derivative thereof.

20. A method for preparing the carrier for cell attachment or fixation, comprising the following steps:
(a) forming a fiber by extruding a melted polymer from a nozzle;
(b) extending the fiber and shaping the extended fiber to form a non-woven structure; and
(c) activating the surface of the non-woven structure to have cell affinity.

21. The method of claim 20 further comprising a step of wrinkling or roughing the surface of the non-woven structure before activating the surface.

22. The method of claim 21, wherein the wrinkled or rough surface of the non-woven structure is formed by a hot pressing step.

23. The method of claim 20, wherein in step (b) the fiber is extended mechanically in a condition of hot air.

24. The method of claim 20, wherein in step (c) the surface of the non-woven structure is activated by an activated grafting treatment.
